SEQUENCE LISTING (3 sequences)

SEQ ID NO: 1 - RNA, 88 nt, Homo sapiens
ugcgcuccucucagucccugagacccuaacuugugauguuuaccguuuaaauccacggguuaggcucuugggagcugcgagucgugcu

SEQ ID NO: 2 - RNA, 89 nt, Homo sapiens
accagacuuuuccuagucccugagacccuaacuugugagguauuuuaguaacaucacaagucaggcucuugggaccuaggcggagggga

SEQ ID NO: 3 - RNA, 22 nt, Homo sapiens
ucccugagacccuaacuuguga

(19) United States
(12) Patent Application Publication
(10) Pub. No.: US 2012/0165208 A1
(43) Pub. Date: Jun. 28, 2012

(54) METHOD FOR PREDICTING THE RESPONSIVENESS OF A PATIENT TO A TREATMENT WITH AN ANTI-CD20 ANTIBODY AND A METHOD FOR DIAGNOSING RHEUMATOID ARTHRITIS

(75) Inventors: Florence Apparailly, Montpellier Cedex (FR); Isabelle Richard, Montpellier Cedex (FR)

(73) Assignee: INSERM (Institut National de la Sante et de la Recherche Medicale), Paris (FR)

(21) Appl. No.: 13/320,054

(22) PCT Filed: May 18, 2010
(86) PCT No.: PCT/EP2010/056765

§ 371 Date: Mar. 14, 2012

(30) Foreign Application Priority Data
May 18, 2009 (EP) 09305447.6

(51) Int. Cl. C12Q 1/68; C40B 30/04

(52) U.S. Cl. 506/9; 435/6.11; 435/6.12

(57) ABSTRACT

The present invention relates to a method for predicting the responsiveness of a patient to a treatment with an anti-CD20 antibody, said method comprising measuring the level of miR-125b expression in a biological sample obtained from said patient. The present invention also relates to a method for diagnosing rheumatoid arthritis in a patient, said method comprising measuring the level of miR-125b expression in a biological sample obtained from said patient.

### FIELD OF THE INVENTION

The present invention relates to a method for predicting the responsiveness of a patient to a treatment with an anti-CD20 antibody, such as rituximab.

### BACKGROUND OF THE INVENTION

Rituximab (RTX) is a human/murine chimeric monoclonal antibody (mAb) that specifically targets the transmembrane protein CD20 of B cells (M. D. Pescovitz, Rituximab, an anti-CD20 monoclonal antibody: history and mechanism of action, Am J Transplant 6 (5) (2006), pp. 859-866.). The binding of RTX to CD20 leads to significant depletion of peripheral B cells (M. E. Reff, K. Carner, K. S. Chambers, P. C. Chinn, J. E. Leonard and R. Raab et al., Depletion of B cells in vivo by a chimeric mouse human monoclonal antibody to CD20, Blood 83 (2) (1994), pp. 435-445; R. P. Taylor and M. A. Lindorfer, Drug insight: the mechanism of action of Rituximab in autoimmune disease—the immune complex decoy hypothesis, Nat Clin Pract Rheumatol 3 (2) (2007), pp. 86-95). RTX sold under the trade names Rituxan® and MabThera® is FDA approved for the treatment of low-grade non-Hodgkin's B cell lymphomas (NHL). Recently it is being increasingly used in the treatment of several autoimmune diseases, such as rheumatoid arthritis.

Rheumatoid arthritis (RA) is a systematic inflammatory autoimmune disorder that affects up to 1% of the European population. RA is characterized by irreversible joint damages, whit disability and ultimately accelerated atherosclerotic cardiovascular and coronary heart disease. Chronic infiltration of the joints by activated immune competent cells including macrophages, T and B cells, together with synovial tissue hyperplasia, leads to cartilage and bone destruction after several years. Although the causes of RA are not fully understood, numerous studies indicate that cytokines are critical in the processes that cause inflammation and joint destruction, TNF-alpha being definitively the prominent one. Currently, in clinic, if diseases activity cannot be controlled with conventional disease modifying anti-rheumatic drugs (DMARD), anti-TNF biotherapies are used. Although a major breakthrough has emerged in the management of RA patients with TNF-alpha blockade, it is not curative and its effects are only partial, non responses common and loss of effect are observed. When patients do no respond to TNF blocking agents (40%), RTX is often prescribed to induce complete remission in the majority of patients.

As consequences, molecular discrimination of responders versus non responders to RTX becomes a major clinical interest, and there is a permanent need in the art for prognostic biomarkers that could assist physicians in providing patients optimized care management with RTX.

### SUMMARY OF THE INVENTION

The present invention relates to a method for predicting the responsiveness of a patient to a treatment with an anti-CD20 antibody, said method comprising measuring the level of miR-125b expression in a biological sample obtained from said patient.

A high level of miR-125b is predictive of a response to an anti-CD20 antibody treatment.

The present invention also relates to a method for diagnosing rheumatoid arthritis in a patient, said method comprising measuring the level of miR-125b expression in a biological sample obtained from said patient.

An increased expression of miR-125b is indicative of rheumatoid arthritis.

### DETAILED DESCRIPTION OF THE INVENTION

The present invention relates to a method for predicting the responsiveness of a patient to a treatment with an anti-CD20 antibody, said method comprising measuring the level of miR-125b expression in a biological sample obtained from said patient.

According to the present invention, “antibody” or “immunoglobulin” have the same meaning, and will be used equally in the present invention. The term “antibody” as used herein refers to immunoglobulin molecules and immunologically active portions of immunoglobulin molecules, i.e., molecules that contain an antigen binding site that immunospecifically binds an antigen. As such, the term antibody encompasses not only whole antibody molecules, but also antibody fragments or derivatives. Antibody fragments include but are not limited to Fv, Fab, F(ab′)2, Fab′, dsFv, scFv, sc(Fv)2 and diabodies.

The term “anti-CD20 antibody” refers to an antibody directed against the CD20 antigen. The CD20 antigen is expressed on B lymphocytes. Examples of anti-CD20 antibodies include but are not limited to rituximab, the yttrium-[90]-labeled 2138 murine antibody designated “Y2B8” (U.S. Pat. No. 5,736,137, expressly incorporated herein by reference); murine IgG2a “131” optionally labeled with ¹³¹I to generate the “¹³¹I-B1” antibody (BEXXARTM®) (U.S. Pat. No. 5,595,721, expressly incorporated herein by reference); murine monoclonal antibody “1F5” (Press et al. Blood 69(2): 584-591 (1987)); “chimeric 2H7” antibody (U.S. Pat. No. 5,677,180 expressly incorporated herein by reference); and monoclonal antibodies L27, G28-2, 93-1133, B-Cl or NU-B2 available from the International Leukocyte Typing Workshop (Valentine et al., In: Leukocyte Typing III (M cMichael, Ed., p. 440, Oxford University Press (1987)). Preferably, said anti-CD20 antibody is rituximab.

The terms “rituximab” “RTX”, or “RITUXAN®” herein refer to the genetically engineered chimeric murine/human monoclonal antibody directed against the CD20 antigen and designated “C2B8” in U.S. Pat. No. 5,736,137, expressly incorporated herein by reference. The antibody is an IgG, kappa immunoglobulin containing murine light and heavy chain variable region sequences and human constant region sequences. Rituximab has a binding affinity for the CD20 antigen of approximately 8.0 nM.

The term “patient” refers to any subject (preferably human) afflicted with a disease likely to benefit from a treatment with an anti-CD20 antibody. Said disease is preferably selected from the diseases that are associated with a proliferation or an over activation of B cells. The diseases may be selected from the group consisting of Hodgkin's B cell lymphomas, non Hodgkin's B cell lymphoma, leukemia; and anto-immune diseases such as rheumatoid arthritis, idiopathic autoimmune hemolytic anemia, Pure red cell aplasia, idiopathic thrombocytopenic purpura, Evans syndrome, vasculitis, multiple sclerosis, bullous skin disorders (for example pemphigus, pemphigoid), type 1 diabetes mellitus, Sjogren's syndrome, Devic's disease and systemic lupus erythematosus.

The term “miRNAs” refers to microRNA molecules that are generally 21 to 22 nucleotides in length, even though lengths of 19 and up to 23 nucleotides have been reported. miRNAs are each processed from a longer precursor RNA molecule (“precursor miRNA”). Precursor miRNAs are transcribed from non-protein-encoding genes. The precursor miRNAs have two regions of complementarity that enables them to form a stem-loop- or fold-back-like structure, which is cleaved in animals by a ribonuclease Ill-like nuclease enzyme called Dicer. The processed miRNA is typically a portion of the stem. The processed miRNA (also referred to as “mature miRNA”) become part of a large complex to down-regulate a particular target gene. All the miRNAs pertaining to the invention are known per se and sequences of them are publicly available from the data base http://microrna.sanger.ac.uk/sequences/.

miR-125b as referred to herein preferably has either: (i) the sequence of the primary transcript of SEQ ID NO:1 or SEQ ID NO:2 or of a sequence with at least 80%, 85%, 90% or 95% or more identity to the sequence of SEQ ID NO:1 or SEQ ID NO:2; or (ii) the sequence of the mature sequence of SEQ ID NO:3 or a sequence with at least 80%, 85%, 90% or 95% or more identity to the sequence of SEQ ID NO:3. There are two hairpin precursors predicted for the mature miR-125b in humans, miR-125b-1 and miR-125b-2, encoded in two different miRNA clusters located in chromosome 11 and 21 respectively²⁴. Therefore the sequences of the primary transcripts of miR-125b are SEQ ID NO:1 for hsa-mir-125b-1 and SEQ ID NO:2 for hsa-mir-125b-2 respectively. The mature sequence of miR-125b is SEQ ID NO:3. The sequence of miR-125b has been deposited at miRBase database under accession number MIMAT0000423.

The term “identity” in the context of nucleic acid sequences refers to the residues in two sequences which are the same when aligned for maximum correspondence. The length of sequence identity comparison may be over a stretch of at least about nine nucleotides, usually at least about 20 nucleotides. There are a number of different algorithms known in the art which can be used to measure nucleotide sequence identity. For instance, polynucleotide sequences can be compared using FASTA, Gap or Bestfit, which are programs in Wisconsin Package Version 10.0, Genetics Computer Group (GCG), Madison, Wis. FASTA provides alignments and percent sequence identity of the regions of the best overlap between the query and search sequences (Pearson, Methods Enzymol. 183:63-98 (1990)). For instance, percent sequence identity between nucleic acid sequences can be determined using FASTA with its default parameters (a word size of 6 and the NOPAM factor for the scoring matrix) or using Gap with its default parameters as provided in GCG Version 6.1. Alternatively, sequences can be compared using the computer program, BLAST (Altschul et al., J. MoI. Biol. 215:403-410 (1990); Gish and States, Nature Genet. 3:266-272 (1993); Madden et al., Meth. Enzymol. 266:131-141 (1996); Altschul et al., Nucleic Acids Res. 25:3389-3402 (1997); Zhang and Madden, Genome Res. 7:649-656 (1997)), especially blastp or tblastn (Altschul et al., Nucleic Acids Res. 25:3389-3402 (1997)).

The terms “biological sample” as used herein refer to a biological sample obtained for the purpose of in vitro evaluation. Typical biological samples to be used in the method according to the invention are blood samples (e.g. whole blood sample or serum sample). In a preferred embodiment, said blood sample is a serum sample. More preferably, the biological sample is a sample obtained from the blood of the patient that contains the non-adherent cell fraction, and more preferably the lymphocyte fraction.

The level of miR-125b expression in the biological sample obtained from the patient may be determined using any technique suitable for detecting RNA levels in a sample. The level of either the primary transcript of miR-125b or the mature sequence of miR-125b, or the level of both the primary transcript and the mature sequence of miR-125b, may be determined. Suitable techniques for determining RNA levels, including miRNA levels, in a biological sample are well known to those skilled in the art and include, for example, Northern blot analysis, PCR techniques such as RT-PCR and real-time RT-PCR, in situ hybridisation, microRNA microarrays, RNAase protection assays, immunological assays or other means. Alternatively, miRNAs quantification method may be performed by using stem-loop primers for reverse transcription (RT) followed by a real-time TaqMan® probe. Typically, said method comprises a first step wherein the stem-loop primers are annealed to miRNA targets and extended in the presence of reverse transcriptase. Then miRNA-specific forward primer, TaqMan® probe, and reverse primer are used for PCR reactions. Quantitation of miRNAs is estimated based on measured CT values. Many miRNA quantification assays are commercially available from Qiagen (S. A. Courtaboeuf, France) or Applied Biosystems (Foster City, USA).

According to a preferred embodiment, the method of the invention is particularly useful to predict the response to a treatment by an anti-CD20 antibody, preferably RTX, in a patient affected with rheumatoid arthritis.

The rheumatoid arthritis can be moderate or active. The disease activity can be measured according to the standards recognized in the art. The “Disease Activity Score” (DAS) is a measure of the activity of rheumatoid arthritis. In Europe the DAS is the recognized standard in research and clinical practice. The following parameters are included in the calculation (Van Gestel A M, Prevoo M L L, van't H of M A, et al. Development and validation of the European League Against Rheumatism response criteria for rheumatoid arthritis. Arthritis Rheum 1996; 39:34-40).
Number of joints tender to the touch (TEN)
Number of swollen joints (SW)
Erythrocyte sedimentation rate (ESR)
Patient assessment of disease activity (VAS; mm)

Patients with a disease activity score 28 (DAS28)>3.2 are a preferred group of patients.

Patients who are resistant to methotrexate (MTX), usually considered first-line therapy for the treatment of RA, are a further preferred group of patients for whom the method of the invention can be particularly useful. More generally, patients who already receive a basic treatment, e.g. with MTX, azathioprine or leflunomide, are particularly good candidates for the test method of the invention. More preferably, patients who already received a TNF blocking agent are particularly good candidates for the test method of the invention.

The method of the invention may further comprise a step of comparing the miR-125b expression level with reference values obtained from responder and non-responder group of patients, wherein detecting differential in the miR-125b expression level between the biological sample and the reference values is indicative whether the patient will be a responder or not to the treatment with the anti-CD20 antibody.

A “responder” patient refers to a patient who shows a clinically significant relief in the disease when treated with an anti-CD20 antibody.

When the disease is RA, a preferred responder group of patients that provides for the control values is a group that shows a decrease of DAS28≧1.2 after three months of treatment with an anti-CD20 antibody, preferably RTX.

After being tested for responsiveness to a treatment with an anti-CD20 antibody, the patients may be prescribed with said anti-CD20 antibody.

A further object of the invention relates to a method for diagnosing rheumatoid arthritis in a patient, said method comprising measuring the level of miR-125b expression in a biological sample obtained from said patient.

The method may further comprise a step consisting of comparing the miR-125b expression level in the biological sample with a reference value, wherein detecting differential in the miR-125b expression level between the biological sample and the reference value is indicative whether the patient is affected with rheumatoid arthritis. According to the invention, the reference value may be obtained from a patient affected with rheumatoid arthritis or from a patient who is not affected with rheumatoid arthritis.

The method of the invention may be used in combination with traditional methods used to diagnose rheumatoid arthritis in a subject.

According to the invention the patients who are affected with rheumatoid arthritis are those who have an increased expression of miR-125b compared to the patients who are not affected, and among the patients who are affected with rheumatoid arthritis, the expression of miR-125b is higher in responders than in non responders.

A further object of the invention relates to a kit for performing the methods of the invention, wherein said kit comprises means for measuring the miR-125b expression in the biological sample obtained from the patient. The kits may include probes, primers as above described.

For example, the kit may comprise a set probes as above defined, usually made of DNA, and that may be pre-labelled. Alternatively, probes may be unlabelled and the ingredients for labelling may be included in the kit in separate containers. The kit may further comprise hybridization reagents or other suitably packaged reagents and materials needed for the particular hybridization protocol, including solid-phase matrices, if applicable, and standards.

Alternatively the kit of the invention may comprise amplification primers (e.g. stem-loop primers) that may be pre-labelled or may contain an affinity purification or attachment moiety. The kit may further comprise amplification reagents and also other suitably packaged reagents and materials needed for the particular amplification protocol.

The invention will be further illustrated by the following figures and example. However, this example and figures should not be interpreted in any way as limiting the scope of the present invention.

### FIGURES

FIG. 1. Deregulated miRNA expression in blood from RA patients. (A) Microarray analysis of miRNAs in blood from RA and healthy donors. Total RNA was extracted from blood samples from 8 RA patients and 8 healthy donors and pooled for miRNA microarray analysis (LC-Sciences). Data are presented on a scatter plot showing log 10-transformed signal intensities for each probe on both channels for the Cy3-labeled control pool (X-axis) and the Cy5-labeled RA pool (Y-axis). (B) Specific over-expression of miR-125b in blood from RA patients. RNA was isolated from blood of healthy individuals (CT, n=13), osteoarthritic patients (OA, n=5) and patients with rheumatoid arthritis (RA, n=16). Mean expression levels of mature miR-125b were quantified by RT-qPCR (Taqman MicroRNA assays). Normalization was performed with small nuclear RNA U6 (RNU6B). The mean values±SD were represented for each group. (C) Expression of miRNAs from miR-125b clusters. miR-100 and miR-99a in blood from healthy individuals (CT, n=7) and patients with rheumatoid arthritis (RA, n=6). Expression levels of miR-100 and miR-99a were quantified by RT-qPCR (Taqman MicroRNA assays). Normalization was performed with small nuclear RNA U6 (RNU6B). *p<0.05, **p<0.01 as determined by Mann-Whitney test.

FIG. 2. High expression levels of miR-125b in sera predict response of RA patients to Rituximab treatment. (A) Specific over-expression of miR-125b in sera from RA patients. RNA was isolated from sera of patients with rheumatoid arthritis (RA, n=35) or osteoarthritic (OA, n=7). (B) Response to anti-CD20 biotherapy. DAS28 M3-M0 represents the difference at baseline versus 3 months after treatment with biologics. Patients were considered non-responders (NR, n=19) or responders (R, n=18) according to EULAR criteria. (C), (D), (E) Differential amounts of 3 microRNAs in the sera of non-responder versus responder RA patients to Rituximab. Sera levels of miR125b (C), miR-142-3p (D), and miR-142-5p (E) were measured individually before initiation of Rituximab treatment. Expression levels of mature miRNAs were quantified by RT-qPCR (Taqman MicroRNA assays). Results were normalized by subtracting the global microRNA level in the sample (average Ct of the 6 microRNAs chosen for normalization) from the levels (Ct) of each microRNA. MicroRNAs miR-142-3p and miR-142-5p levels are not significantly different. *p<0.05, **p<0.005 were determined by Mann-Whitney test.

FIG. 3. Up-regulation of miR-125b expression in activated cell types. (A) Induction fold expression of miR-125b expression in human T cell line Jurkat (T cells), B cell line Daudi (B cells) and monocytes cell line THP1 (Monocytes) following inflammatory challenge. The 3 different human cell lines were stimulated (50 ng/ml) with either TNF-α (black bars) or IL1-β (white bars) for 4 hours. The miR-125b expression was analysed by RT-qPCR (Taqman MicroRNA assays). Normalization was performed with small nuclear RNA U6 (RNU6B). The mean valuse±SD were represented for each group. (B) Lymphocytes fraction is the major contributor to miR-125b over-expression detected in blood. PBMCs were collected from RA patients (n=5) and separated into monocyte/macrophage (Adh.) and lymphocyte (N-adh) populations by allowing the monocytes/macrophages to adhere to tissue culture dish.

FIG. 4. Enforced miR-125b expression modifies the cytokine secretion profile of activated T lymphocytes. (A) Primary T cell lines from RA patients were transiently transfected with scrambled miRNA precursor (white bars) or miR-125b precursor or pre-miR-125b (black bars) by nucleofection Amaxa technique. Total RNA was extracted with mirVana miRNA Isolation Kit. Mean expression levels of mature miR-125b were quantified by RT-qPCR (Taqman MicroRNA assays) and normalization with small nuclear RNA U6 (RNU6B). (B) Pro- and anti-inflammatory cytokine secretion profile of activated primary human T cells. Primary T cell line were Transiently transfected with different concentration of pre-miR-125b (hatched bars represent miR-125b low, 20 pmol; green bars, miR-125b high, 100 pmol), and were with plate-bound anti-CD3 and soluble anti-CD28 mAb. Secretion of IL-17, IL-22, TNF-α, IL-4 and INF-γ were quantified by ELISA. The mean values±SD were represented for each group.

### EXAMPLE

### Microrna-125B Over-Expression in Serum of Patients with Rheumatoid Arthritis Predicts Responsiveness to Rituximab

Material & Methods

Patients and healthy controls: Fresh peripheral blood and serum samples were obtained from healthy donors with no history of autoimmune diseases and patients with osteoarthritis (OA) or RA fulfilling the 1987 revised classification criteria of the American College of Rheumatology²⁰, followed in the rheumatology department at the university hospital of Montpellier (France). Patients were assessed for overall disease activity using the 28-joint-count Disease Activity Score (DAS28) as previously described²¹. The criteria for patient eligibility were: methotrexate (MTX) treatment; DAS28≧5.1; and resistance to at least 2 DMARDs (MTX and anti-TNF included). For one month or more before the start of this study, every patient was given stable doses of oral corticosteroids and did not receive any intra-articular steroid injections. Patients were treated with rituximab (MabThera®, Roche) as recommended by the manufacturer and the French Drug Agency AFSSAPS (intravenously 1,000 mg one time at day 0, and day 15). RA patients were separated in two sub-groups according to their clinical response to the rituximab after 3 months (M3) of treatment (DAS28 M3-M0), following the EULAR criteria: for non-responders (NR), DAS28>5.1 and the ratio DAS28 M3-M0≦0.6; for responders (R), DAS28 M3-M0>1.2.

Blood RNA isolation: Blood samples were collected using EDTA-coated tubes (BD Vacutainer™ 5 ml; BD Diagnostics, France) according to standard procedure. Aliquots of 0.5 ml of blood samples were immediately transferred to 1.2 ml of RNAlater medium (Applied Biosystems) and stored at −20° C. Total RNA was extracted using a modified protocol from the Ribopure-Blood RNA isolation kit (Applied Biosystems). Briefly, 10 μl glacial acid (Sigma, France) was added to blood cell lysate (800 μl, step 1 and 2 according to the manufacturer's instruction). The samples were extracted with acid phenol/Chloroform, 1 ml of GuSCN Lysis solution (4 M Guanidinium Thiocyanate, 25 mM Sodium Citrate, 0.5% (w/v) Sodium N-lauroyl Sarcosinate and 0.1 M beta-mercaptoethanol and 1.25 volumes of ethanol were added to the aqueous phase. The samples were passed through a Filter cartridge and washed, first with wash solution 1 (70% EtOH/30% GuSCN lysis solution) and second with wash solution 2 (80% EtOH/50 mM NaCl). The RNA was eluted in 100 μl Elution solution preheated to 80° C. and stored at −20° C. The concentration and integrity of RNA were determined by NanoDrop ND-1000 spectrophotometry (NanoDrop Tech, Rockland, Del) and by a Bioanalyser Agilent 1. For pool samples, measures were taken to guarantee that RNA from each subject was of equal amount.

MicroRNA microarray: Total RNA was extracted from blood samples from 8 RA patients and 8 healthy donors as explained above, and pooled for miRNA microarray analysis. RNA quality control, labelling, hybridization, and scanning were performed by LC Sciences (Houston, Tex.) using the latest probe content in the Sanger miRBase (Sanger miRBase 11.0). Three microarray experiments were performed. Preliminary statistical analysis was performed by LC sciences on raw data normalized by the locally weighted regression method on the background-subtracted data. Further statistical comparisons were performed using analysis of variance. miRNAs that were modulated>±0.5-fold with a P value of <0.01 were considered significant.

Real-time quantitative reverse transcription PCR (RT-qPCR): For miRNAs analysis, 10 ng of total RNA was reverse transcribed using 50 nM human microRNA specific stem-loop RT primers, 50 units/μl MultiScribe reverse transcriptase, 10XRT buffer, 100 mM each dNTPs, and 20 units/μl RNase inhibitor (Applied Biosystems). Reaction mixtures (15 μl) were incubated in a thermocycler Mastercycler (Eppendorf, France) for 30 minutes at 16° C., 30 minutes at 42° C., 5 minutes at 85° C. and then maintained at 4° C. Real-time PCR was performed on the resulting complementary DNA using TaqMan microRNA specific primers and TaqMan Universal PCR Master Mix. All the experiments were performed according to the manufacturer's protocols, using a pipeting robotic platform epMotion 5070 (Eppendorf) and a LightCycler 480 Detection system (Roche, France). The expression of the U6B small nuclear RNA (RNU6B) was used as endogenous control for data normalization. Relative expression was calculated using the comparative threshold cycle (Ct) method.

RNA sera extraction and quantification by RT-qPCR: Whole blood was separated into serum and cellular fractions within 2 h following collection. Sera were stored at −20° C. RNA extraction of 400 μl serum was performed by acid phenol:chloroform extraction and precipitated with ethanol over-night at −20° C.¹⁸. After precipitation, 40 μl of sterile water was added to the RNA isolation. Typically, a 15 μl reverse transcriptase reaction contained 6.7 μl of purified RNA and reverse transcription was performed according to the manufacturer's instruction. Real-time PCR was performed on the resulting complementary DNA using TaqMan microRNA specific primers and TaqMan Universal PCR Master Mix. Since U6 and 5S rRNA were degraded in serum samples^{18,19}, results were normalized by subtracting the global miRNA levels in the sample (average Cₜ of the 6 miRNAs, hsa-miR-142-3p, hsa-miR-142-5p, hsa-miR-24, hsa-miR-181d, hsa-miR-15b and hsa-miR-125b) from the level (Cₜ) of each miRNA¹⁸.

Cell culture and RNA isolation: The human monocytic THP-1 cell line, B cell line DAUDI and T cell line JURKAT were grown in RPMI medium (Gibco, France) supplemented with 10% FBS, 1× nonessential amino acids, 100 units/ml penicillin, 100 units/ml streptomycin and 2 mM glutamine in a humidified incubator containing 5% CO₂ at 37° C. Cells were seeded at 1.5×10⁵/well into a 24 well-plate containing 1 ml of supplemented medium and incubated for 24 hours. Cells were stimulated or not with 50 ng/ml of human TNF-α or IL1-β for 4 hours.

The primary T lymphocyte lines, generated from RA patients²², were grown in Yssel's medium supplemented with 1% human AB+ serum and 2 ng/ml rIL-2 (R&D Systems). Stimulation of cells was performed using anti-CD3 mAb immobilized onto plastic tissue culture plates and soluble (1 μg/ml) anti-CD28 mAb²³. For each nucleofection assay, the primary T cell lines were numbered, 6-8×10⁶ cells were suspended in 100 μl Nucleofector buffer (Human Monocyte Nucleofector kit VPA-1008, Amaxa Bioscience, Germany), and nucleofected either with 2 μg of pmaxGFP plasmid or 20-100 pmol of pre-miR™-125b and pre-miR™-CTRL (Applied Biosystem). Transfections were performed using program U-014 according to manufacturer's instructions. Cells were then plated in 24 well-plates for RNA extractions and in 96 well-plates for ELISA. Supernatants for cytokine detections and cells for miRNA analyses were collected 48 hours and 6 hours after activation with anti-CD3/CD28 mAb procedure respectively.

Primary lymphocyte and monocyte/macrophage fraction: Blood samples were collected in heparine-treated tubes and PBMCs were isolated by standard Ficoll density-gradient centrifugation. PBMCs were washed once in sterile phosphate buffered saline (PBS) before culture. Monocyte/macrophage and lymphocytes populations were separated by allowing the monocytes/macrophages to adhere to a tissue culture dish. For all RNA purification, cells were washed twice with cold PBS, and total RNA was isolated with mirVana RNA isolation kit (Applied Biosystems, France) according to the manufacturer's instruction. Cell supernatants were stored at −20° C. until assayed. The human IL-4, IL-17, IL-22, IFN-γ and TNF-α secretion were measured by specific ELISA kits (CliniSciences, France) according to the manufacturer's protocol.

Statistical analysis: Data were analysed statistically using the Mann-Whitney U test. Analyses were performed using the http://www.viesanimales.org website. P values less than 0.05 were considered statistically significant. The Power and Precision V3 Software (http://ww.power-anaylsis.com) was used to calculate the 1-β error (the probability of a p=2α<0.01 not appearing at random) for the difference in sera levels of mir-125b between responders and non-responders.

Results

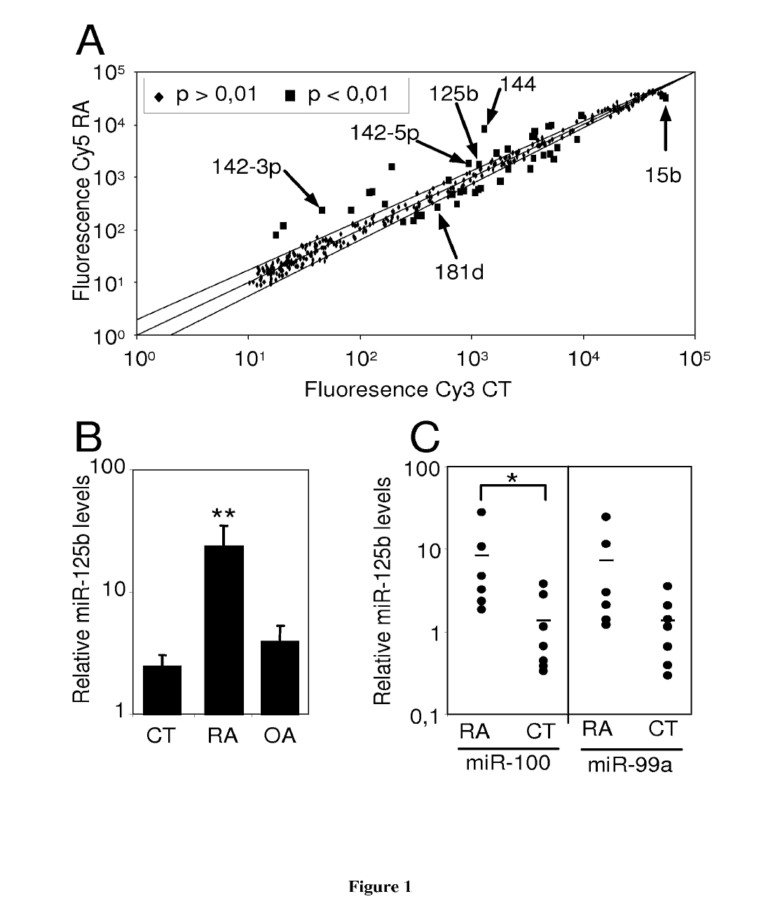

Increased expression of miR-125b in blood from RA patients: A genome-wide miRNA expression profiling was performed using a subtractive approach to identify miRNAs differentially expressed between normal and RA blood samples. Total RNAs were isolated from 0.5 ml of RA and healthy whole blood samples, and pooled (n=8) for analysis of the expression levels of 723 unique human miRNAs in their mature forms (FIG. 1A). The RA samples were collected at disease flare from patients having similar clinical features satisfying the ACR criteria²⁰: ACPA positive and DAS28>5.1. A total of 37 miRNAs were differentially expressed (p<0.01), with 20 miRNAs up-regulated and 17 miRNAs down-regulated in RA patients compared with healthy donors. To confirm the miRNA micro-array expression data, levels of 6 miRNAs differentially expressed were measured on a larger cohort of samples using RT-qPCR analysis: miR-15b, miR-125b, miR-142-3p, miR-142-5p, miR-144 and miR-181d. Data from the miRNA microarray screen were confirmed for 5 out of 6 miRNAs, only miR-144 over-expression was not reproduced. Remarkably, miR-125b was significantly over-expressed in patients with full-blown RA (n=16) as compared with healthy controls (n=13) (FIG. 1B). To determine whether miR-125b up-regulation was specific for RA, blood samples from osteoarthritic (OA) patients were also analysed. The expression levels of miR-125b in OA blood were not significantly different from those in controls (FIG. 1B).

There are two hairpin precursors predicted for the mature miR-125b in humans, miR-125b-1 and miR-125b-2, encoded in two different miRNA clusters located in chromosome 11 and 21 respectively²⁴. To determine whether the increased mature miR-125b expression observed was preferentially related to the up-regulation of one of these 2 miRNA clusters, we analyzed the levels of one miRNA encoded in each cluster by RT-qPCR. Both miR-99 and miR-100 were similarly over-expressed in blood from RA patients compared with healthy donors (FIG. 1C), suggesting that both clusters are similarly deregulated in RA. Our data reinforce the idea that a subset of miRNAs is aberrantly expressed in RA and thus might provide the first evidence of novel regulators specifically involved in RA pathogenesis and of promising potential as non invasive miRNA-based diagnosis biomarkers.

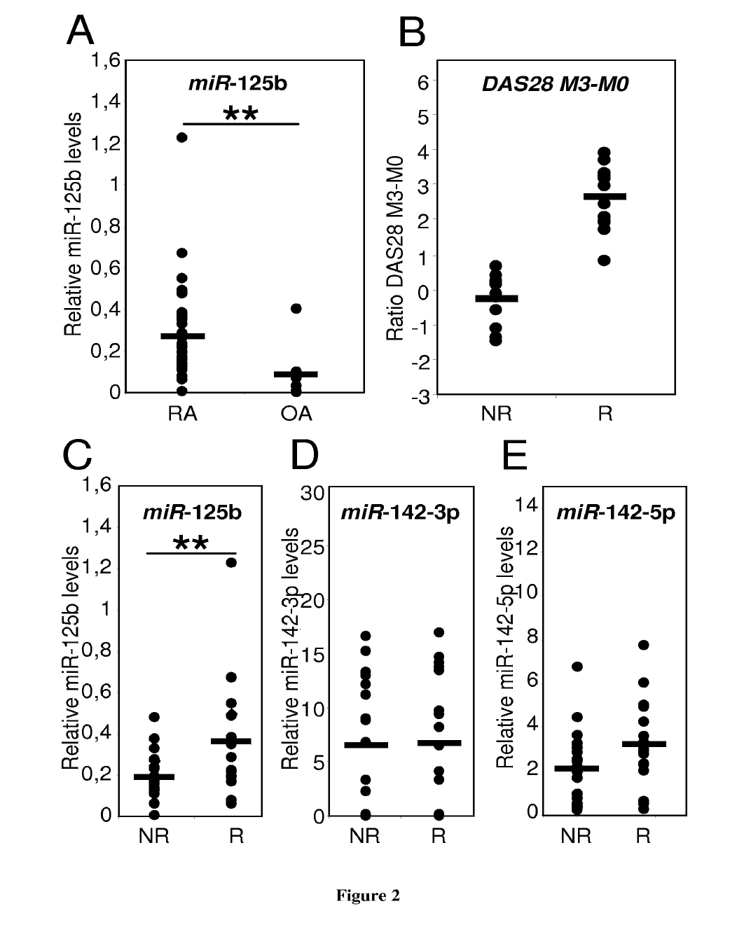

High expression levels of miR-125b in RA serum correlate with a positive response to rituximab therapy: Using a previously reported approach for the direct detection of some miRNAs in sera^{18,19}, we next investigated whether miR-125b up-regulation could also be measured in the sera of patients with active RA and whether its expression level could be used as biomarker to predict clinical responses to current biologics such as rituximab (FIG. 2). Indeed, miR-125b was detectable by RT-qPCR in sera, and its increased expression levels observed in patients with RA in comparison with control and OA subjects was confirmed (FIG. 2A). Most importantly, when RA patients were divided in two sub-groups according to their clinical response to the rituximab after 3 months of treatment, DAS28 M3-M0 (FIG. 2B), results showed that high expression of miR-125b was associated with a good response to anti-CD20 therapy (FIG. 2C). Indeed, serum levels of miR-125b before the initiation of treatment were substantially higher in good responders compared with non responders, while two other miRNAs also deregulated in RA, namely miR-142-3p and miR-142-5p, were not expressed at significantly different levels in both groups of patients (FIGS. 2D and 2E). These data suggest that RA patients with low expression of miR-125b at the time of disease flare have significantly lower chance to improve clinically after 3 months of rituximab treatment and that serum abundance of miR-125b could be used as predictive biomarker. With mean value 0.36±0.26 for responders (n=18) and 0.19±0.12 for non-responders (n=19), the power analysis yielded a 1-β value of 71%. By increasing the number of patients to only 40 in each treatment group, keeping the difference between means and the SD-values constant, the power analysis gave a 1-β value close to 100%. This signifies that an analysis of a single sera sample for mir-125b contents will serve as a very good predictor or clinical marker for a patient's response to treatment.

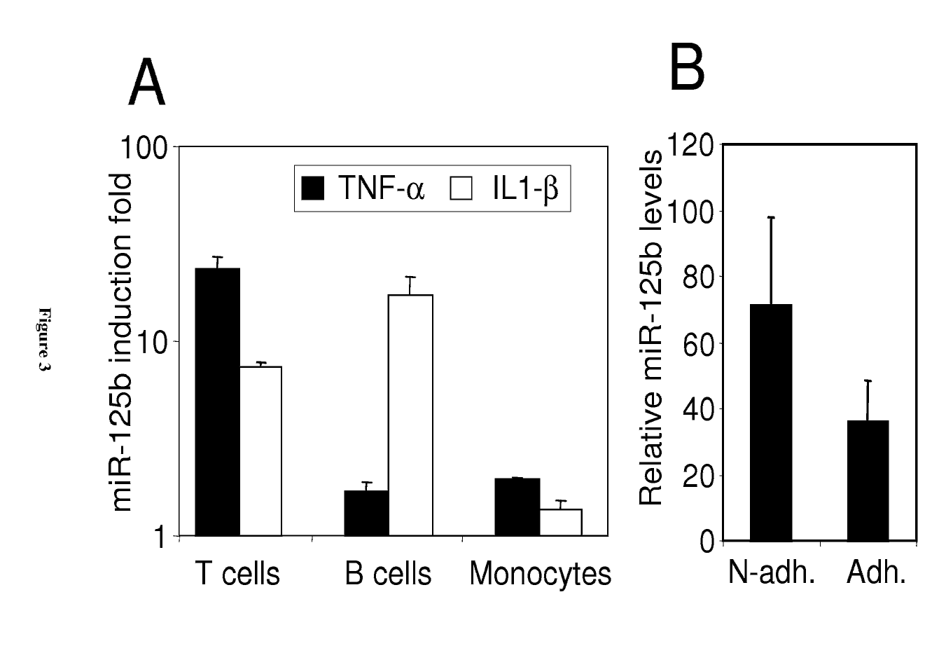

Identification of cells responsible for the miR-125b over-expression in RA blood: Microarray and RT-qPCR analyses were based on whole blood or sera samples and therefore reflect different cellular contributions. To investigate which specific cell types expressed miR-125b, we first examined the changes in miR-125b expression levels in various human blood cell lines (FIG. 3A). The expression of miR-125b was determined by RT-qPCR in the T cell line Jurkat, the B Burkitt lymphoma cell line Daudi, and the THP1 monocytic cell line, 4 hours after stimulation with TNF-α or IL-1β. Although miR-125b expression was increased in all conditions, the strongest induction following pro-inflammatory challenge was obtained in lymphocytes; mainly in T lymphocytes after TNF-stimulation, and in T and B lymphocytes following IL-1β stimulation. These data were confirmed on primary cells from RA patients (n=5), as miR-125b up-regulation was mainly attributable to the non-adherent cell fraction (lymphocyte population) versus the adherent cell fraction (monocyte/macrophage population). RT-qPCR quantification revealed a 2-fold difference in miR-125b abundance between both fractions (FIG. 3B). Taken together, our findings document an increased expression of miR-125b in all circulating immune cells under inflammatory conditions, with a predominant contribution of lymphocytes.

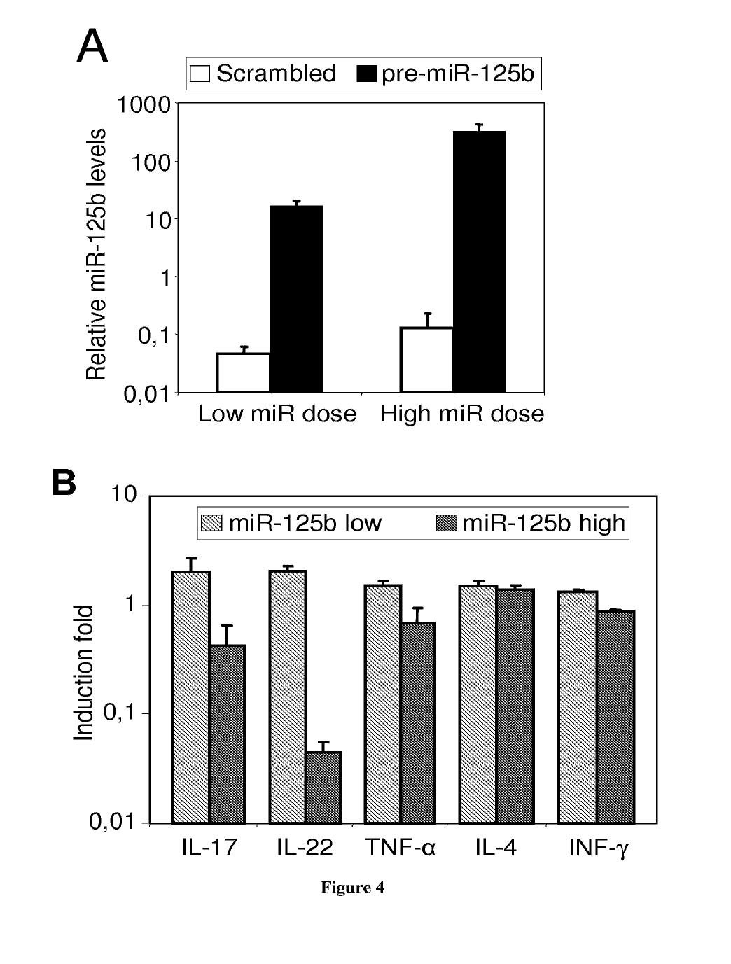

Over-expression of miR-125b in lymphocytes modulates a pro-inflammatory cytokine profile: To evaluate the functional consequences of the deregulated miR-125b expression in RA lymphocytes, T lymphocyte lines were generated from RA patients and miR-125b was over-expressed using two different doses of pre-miR-125b (20 and 100 pmol). As assessed by flow cytometry, 37% of T cells were efficiently transfected 48 hours post-nucleofection with a GFP-encoding plasmid DNA and dose-dependent miR-125b over-expression was confirmed by RT-qPCR (FIG. 4A). Compared with control scrambled pre-miRNA, expression levels of miR-125b were increased by log 2.5- and log 3.5-fold in T lymphocytes transfected with low and high doses of pre-miR-125b respectively. Since the 3′-UTR of TNF-α transcript has been validated as a miR-125b target²⁵, TNF-α expression was quantified at both mRNA and protein levels following T cell activation, as well as other T lymphocyte-derived cytokines (FIG. 4B). The levels of TNF-a transcript were unchanged, while the protein levels were decreased following high dose of pre-miR-125b transfection (FIG. 4B), suggesting that the miR-125b does not affect TNF-α mRNA stability. Interestingly, strong induction of miR-125b expression in activated T lymphocytes secreting predominantly IFN-γ and IL-17 also resulted in a decreased production of IFN-γ, IL-17 and IL-22, while IL-4 expression was unchanged, whereas moderate up-regulation of miR-125b had only little effect on the cytokine production profile (FIG. 4B). Thereafter, these data support the notion that miR-125b plays an important role in the regulation of cytokine network in T lymphocytes, in particular by dampening the production of signature cytokines of both Th1 and Th17 cells.

### CONCLUSIONS

Our findings show that systemic expression of miR-125b can be used as potential diagnostic biomarker for RA and, most importantly, that high serum levels of miR-125b at disease flares can predict for rituximab biotherapy success, and thus might be used as prognostic biomarker as well. Indeed, sera levels of miR-125b were substantially higher in good responders compared with non responders before the initiation of the anti-CD20 biotherapy.

### REFERENCES

Throughout this application, various references describe the state of the art to which this invention pertains. The disclosures of these references are hereby incorporated by reference into the present disclosure.
1. Klareskog L, Catrina A I, Paget S. Rheumatoid arthritis. Lancet. 2009; 373:659-672.
2. Sokka T, Abelson B, Pincus T. Mortality in rheumatoid arthritis: 2008 update. Clin Exp Rheumatol. 2008; 26:S35-61.
3. Bongartz T, Sutton A J, Sweeting M J, Buchan I, Matteson E L, Montori V. Anti-TNF antibody therapy in rheumatoid arthritis and the risk of serious infections and malignancies: systematic review and meta-analysis of rare harmful effects in randomized controlled trials. Jama. 2006; 295:2275-2285.
4. Fabre S, Dupuy A M, Dossat N, et al. Protein biochip array technology for cytokine profiling predicts etanercept responsiveness in rheumatoid arthritis. Clin Exp Immunol. 2008; 153:188-195.
5. van Venrooij W J, van Beers J J, Pruijn G J. Anti-CCP Antibody, a Marker for the Early Detection of Rheumatoid Arthritis. Ann N Y Acad. Sci. 2008; 1143:268-285.
6. Lequerre T, Gauthier-Jauneau A C, Bansard C, et al. Gene profiling in white blood cells predicts infliximab responsiveness in rheumatoid arthritis. Arthritis Res Ther. 2006; 8:R105.
7. Pillai R S, Bhattacharyya S N, Filipowicz W. Repression of protein synthesis by miRNAs: how many mechanisms? Trends Cell Biol. 2007; 17:118-126.
8. Lodish H F, Zhou B, Liu G, Chen C Z. Micromanagement of the immune system by microRNAs. Nat Rev Immunol. 2008; 8:120-130.
9. Sonkoly E, Stahle M, Pivarcsi A. MicroRNAs and immunity: novel players in the regulation of normal immune function and inflammation. Semin Cancer Biol. 2008; 18:131-140.
10. Tili E, Michaille J J, Costinean S, Croce C M eds. MicroRNAs, the immune system and rheumatic disease. Nat Clin Pract Rheumatol; 2008.
11. Lindsay M A. microRNAs and the immune response. Trends Immunol. 2008; 29:343-351.
12. Bhanji R A, Eystathioy T, Chan E K, Bloch D B, Fritzler M J. Clinical and serological features of patients with autoantibodies to GW/P bodies. Clin Immunol. 2007; 125:247-256.
13. Sonkoly E, Stahle M, Pivarcsi A. MicroRNAs: novel regulators in skin inflammation. Clin Exp Dermatol. 2008; 33:312-315.
14. Dai Y, Huang Y S, Tang M, et al. Microarray analysis of microRNA expression in peripheral blood cells of systemic lupus erythematosus patients. Lupus. 2007; 16:939-946.
15. Stanczyk J, Pedrioli D M, Brentano F, et al. Altered expression of MicroRNA in synovial fibroblasts and synovial tissue in rheumatoid arthritis. Arthritis Rheum. 2008; 58:1001-1009.
16. Nakasa T, Miyaki S, Okubo A, et al. Expression of microRNA-146 in rheumatoid arthritis synovial tissue. Arthritis Rheum. 2008; 58:1284-1292.
17. Pauley K M, Satoh M, Chan A L, Bubb M R, Reeves W H, Chan E K. Upregulated miR-146a expression in peripheral blood mononuclear cells from rheumatoid arthritis patients. Arthritis Res Ther. 2008; 10:R101.
18. Gilad S, Meiri E, Yogev Y, et al. Serum microRNAs are promising novel biomarkers. PLoS ONE. 2008; 3:e3148.
19. Mitchell P S, Parkin R K, Kroh E M, et al. Circulating microRNAs as stable blood-based markers for cancer detection. Proc Natl Acad Sci USA. 2008; 105:10513-10518.
20. Arnett F C, Edworthy S M, Bloch D A, et al. The American Rheumatism Association 1987 revised criteria for the classification of rheumatoid arthritis. Arthritis Rheum. 1988; 31:315-324.
21. Fransen J, van Riel P L. The Disease Activity Score and the EULAR response criteria. Clin Exp Rheumatol. 2005; 23:S93-99.
22. Pene J, Chevalier S. Preisser L, et al. Chronically inflamed human tissues are infiltrated by highly differentiated Th17 lymphocytes. J. Immunol. 2008; 180:7423-7430.
23. Pene J, Rahmoun M, Temmerman S, Yssel H. Use of anti-CD3/CD28 mAb coupled magnetic beads permitting subsequent phenotypic analysis of activated human T cells by indirect immunofluorescence. J Immunol Methods. 2003; 283:59-66.
24. Lee Y S, Kim H K, Chung S, Kim K S, Dutta A. Depletion of human micro-RNA miR-125b reveals that it is critical for the proliferation of differentiated cells but not for the down-regulation of putative targets during differentiation. J Biol Chem. 2005; 280:16635-16641.
25. Tili E, Michaille J J, Cimino A, et al. Modulation of miR-155 and miR-125b levels following lipopolysaccharide/TNF-alpha stimulation and their possible roles in regulating the response to endotoxin shock. J Immunol. 2007; 179:5082-5089.
26. Yendamuri S, Calin G A. The role of microRNA in human leukemia: a review. Leukemia. 2009.
27. Carissimi C, Fulci V, Macino G. MicroRNAs: Novel regulators of immunity. Autoimmun Rev. 2009.
28. Resnick K E, Alder H, Hagan J P, Richardson D L, Croce C M, Cohn D E. The detection of differentially expressed microRNAs from the serum of ovarian cancer patients using a novel real-time PCR platform. Gynecol Oncol. 2009; 112:55-59.
29. Hunter M P, Ismail N, Zhang X, et al. Detection of microRNA expression in human peripheral blood microvesicles. PLoS ONE. 2008; 3:e3694.
30. Malumbres R, Sarosiek K A, Cubedo E, et al. Differentiation-stage-specific expression of microRNAs in B-lymphocytes and diffuse large B-cell lymphomas. Blood. 2008.
31. Le M T, Teh C, Shyh-Chang N, et al. MicroRNA-125b is a novel negative regulator of p53. Genes Dev. 2009.
32. Sempere L F, Freemantle S, Pitha-Rowe I, Moss E, Dmitrovsky E, Ambros V. Expression profiling of mammalian microRNAs uncovers a subset of brain-expressed microRNAs with possible roles in murine and human neuronal differentiation. Genome Biol. 2004; 5:R13.
33. Mizuno Y, Yagi K, Tokuzawa Y, et al. miR-125b inhibits osteoblastic differentiation by down-regulation of cell proliferation. Biochem Biophys Res Commun. 2008; 368:267-272.
34. Yamada H, Yanagisawa K, Tokumaru S, et al. Detailed characterization of a homozygously deleted region corresponding to a candidate tumor suppressor locus at 21q11-21 in human lung cancer. Genes Chromosomes Cancer. 2008; 47:810-818.
35. Bousquet M, Quelen C, Rosati R, et al. Myeloid cell differentiation arrest by miR-125b-1 in myelodysplastic syndrome and acute myeloid leukemia with the t(2;11)(p21;q23) translocation. J Exp Med. 2008; 205:2499-2506.
36. Asirvatham A J, Magner W J, Tomasi T B. miRNA regulation of cytokine genes. Cytokine. 2009; 45:58-69.
37. Brustle A, Heink S, Huber M, et al. The development of inflammatory T(H)-17 cells requires interferon-regulatory factor 4. Nat Immunol. 2007; 8:958-966.
38. O'Connell R M, Taganov K D, Boldin M P, Cheng G, Baltimore D. MicroRNA-155 is induced during the macrophage inflammatory response. Proc Natl Acad Sci USA. 2007; 104:1604-1609.
39. Xiao C, Rajewsky K. MicroRNA control in the immune system: basic principles. Cell. 2009; 136:26-36.
40. Wu H, Neilson J R, Kumar P, et al. miRNA profiling of naive, effector and memory CD8 T cells. PLoS ONE. 2007; 2:e1020.
41. Huang B, Zhao J, Lei Z, et al. miR-142-3p restricts cAMP production in CD4(+)CD25(−) T cells and CD4(+)CD25(+) T(REG) cells by targeting AC9 mRNA. EMBO Rep. 2008.
42. Calin G A, Dumitru C D, Shimizu M, et al. Frequent deletions and down-regulation of micro-RNA genes miR15 and miR16 at 13q14 in chronic lymphocytic leukemia. Proc Natl Acad Sci USA. 2002; 99:15524-15529.
43. Bottoni A, Piccin D, Tagliati F, Luchin A, Zatelli M C, degli Uberti EC. miR-15a and miR-16-1 down-regulation in pituitary adenomas. J Cell Physiol. 2005; 204:280-285.
44. Cimmino A, Calin G A, Fabbri M, et al. miR-15 and miR-16 induce apoptosis by targeting BCL2. Proc Natl Acad Sci USA. 2005; 102:13944-13949.
45. Li Q J, Chau J, Ebert P J, et al. miR-181a is an intrinsic modulator of T cell sensitivity and selection. Cell. 2007; 129:147-161.
46. de Yebenes V G. Belver L, Pisano D G, et al. miR-181b negatively regulates activation-induced cytidine deaminase in B cells. J Exp Med. 2008; 205:2199-2206.
47. Naguibneva I, Ameyar-Zazoua M, Polesskaya A, et al. The microRNA miR-181 targets the homeobox protein Hox-A 11 during mammalian myoblast differentiation. Nat Cell Biol. 2006; 8:278-284.
48. Kuhn D E, Nuovo G J, Martin M M, et al. Human chromosome 21-derived miRNAs are overexpressed in down syndrome brains and hearts. Biochem Biophys Res Commun. 2008; 370:473-477.
49. Mease P J. B cell-targeted therapy in autoimmune disease: rationale, mechanisms, and clinical application. J. Rheumatol. 2008; 35:1245-1255.
50. Lawrie C H, Gal S, Dunlop H M, et al. Detection of elevated levels of tumour-associated microRNAs in serum of patients with diffuse large B-cell lymphoma. Br J Haematol. 2008; 141:672-675.

1. A method for predicting the responsiveness of a patient to a treatment with an anti-CD20 antibody, said method comprising measuring the level of miR-125b expression in a biological sample obtained from said patient.

2. The method according to claim 1 wherein said miR-125b is chosen from the group consisting of the primary transcript sequences SEQ ID NO:1 and SEQ ID NO:2, the mature sequence SEQ ID NO:3, and the sequences with at least 80% identity to one of the sequences of SEQ ID NO:1, SEQ ID NO:2 and SEQ ID NO:3.

3. The method according to claim 1 wherein said anti-CD20 antibody is rituximab.

4. The method according to claim 1, wherein said patient is affected with rheumatoid arthritis.

5. The method according to claim 4, wherein said patient is affected with rheumatoid arthritis that is active.

6. The method according to claim 5 wherein said patient has been already treated with a TNF blocking agent.

7. The method according to claim 1 which further comprises a step of comparing the miR-125b expression level with reference values obtained from responder and non-responder group of patients.

8. A method for diagnosing rheumatoid arthritis in a patient, said method comprising measuring the level of miR-125b expression in a biological sample obtained from said patient.

9. The method according to claim 1, wherein said biological sample is a blood sample.

10. The method according to claim 2, wherein said anti-CD20 antibody is rituximab.

11. The method according to claim 2, wherein said patient is affected with rheumatoid arthritis.

12. The method according to claim 3, wherein said patient is affected with rheumatoid arthritis.

13. The method according to claim 2, which further comprises a step of comparing the miR-125b expression level with reference values obtained from responder and non-responder group of patients.

14. The method according to claim 3, which further comprises a step of comparing the miR-125b expression level with reference values obtained from responder and non-responder group of patients.

15. The method according to claim 4, which further comprises a step of comparing the miR-125b expression level with reference values obtained from responder and non-responder group of patients.

16. The method according to claim 5, which further comprises a step of comparing the miR-125b expression level with reference values obtained from responder and non-responder group of patients.

17. The method according to claim 6, which further comprises a step of comparing the miR-125b expression level with reference values obtained from responder and non-responder group of patients.

18. The method according to claim 8, wherein said biological sample is a blood sample.
